(19) United States
(12) Patent Application Publication
(10) Pub. No.: US 2014/0179958 A1
(43) Pub. Date: Jun. 26, 2014

(54) CATALYSTS AND PROCESSES FOR PRODUCING BUTANOL

(71) Applicant: CELANESE INTERNATIONAL CORPORATION, Irving, TX (US)

(72) Inventor: Cheng Zhang, Houston, TX (US)

(73) Assignee: CELANESE INTERNATIONAL CORPORATION, Irving, TX (US)

(21) Appl. No.: 13/722,023

(22) Filed: Dec. 20, 2012

(51) Int. Cl. C07C 29/34; B01J 23/62; B01J 23/89; B01J 23/58

(52) U.S. Cl. CPC C07C 29/34; B01J 23/58; B01J 23/624; B01J 23/8966
USPC 568/905; 502/328; 502/326

(57) ABSTRACT

In one embodiment, the invention is to a process for producing a catalyst composition for converting ethanol to higher alcohols, such as butanol. The process comprises contacting magnesium carbonate with one or more metal precursors to form a catalyst intermediate and calcining the catalyst intermediate to form the catalyst composition that comprises the one or more metals and magnesium oxide. The one or more metal precursors comprises one or more metal selected from the group consists of nickel, palladium, platinum, germanium, copper, ruthenium, gallium, tin, iridium, and mixtures thereof.

### FIELD OF THE INVENTION

The present invention relates generally to a process of making higher molecular weight alcohols from ethanol and, in particular, to a catalytic conversion of ethanol to butanol.

### BACKGROUND OF THE INVENTION

Studies have been done for economically viable processes to produce butanol. Like ethanol, butanol may be a possible solution to dependency on oil as both may be used as a fuel in an internal combustion engine. In fact, due to the longer hydrocarbon chain and non-polar characteristics, butanol may be a better fuel option than ethanol because butanol is more similar to gasoline than ethanol. In addition, butanol may be used in the manufacture of pharmaceuticals, polymers, pyroxylin plastics, herbicide esters and butyl xanthate. Butanol may also be used as a solvent for the extraction of essential oils or as an ingredient in perfumes; as an extractant in the manufacture of antibiotics, hormones, and vitamins; as a solvent for paints, coatings, natural resins, gums, synthetic resins, alkaloids, and camphor. Other applications of butanol includes as swelling agent in textiles; as a component of break fluids, cleaning formulations, degreasers, and repellents; and as a component of ore floatation agents and of wood-treating systems.

Butanol is typically produced industrially from petrochemical feedstock propylene in the presence of a rhodium-based homogeneous catalyst. During this process, propylene is hydroformylated to butyraldehyde and butyraldehyde is then hydrogenated to product butanol. However, due to the fluctuating natural gas and crude oil prices the cost of producing butanol using this method also becomes more unpredictable and significant.

It is known that butanol may be prepared by condensation from ethanol over basic catalyst at high temperature using the Guerbet reaction. The reaction mechanism for the conversion of ethanol to butanol via the Guerbet reaction comprises a four-step sequence as shown in reaction scheme 1. In the first step, ethanol is oxidized to intermediate aldehyde and two of the intermediate aldehydes undergo an aldol condensation reaction to form crotonaldehyde, which is reduced to butanol via hydrogenation. See, for example, J. Logsdon in Kirk-othmer Encyclopedia of Chemical Technology, John Wiley and Sons, Inc., New York, 2001; J. Mol. Catal. A: Chem., 2004, 212, p.65; and J. Org. Chem., 2006, 71, p. 8306.

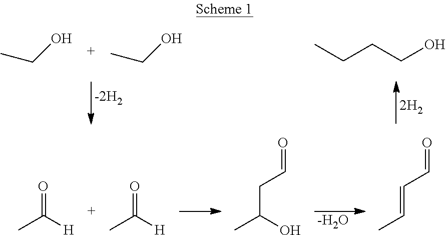

Various catalysts have been studied to improve the conversion and selectivity of ethanol to butanol. For example, M. N. Dvornikoff and M. W. Farrar, J. of Organic Chemistry (1957), 11, 540-542, discloses the use of a MgO—K₂CO₃—CuCrO₂ catalyst system to promote ethanol condensation to higher alcohols, including butanol. U.S. Pat. No. 5,300,695 discloses processes where an L-type zeolite catalyst, potassium L-type zeolite, is used to react with an alcohol having X carbon atoms to produce alcohol with higher molecular weight.

The use of hydroxyapatite Ca₁₀(PO₄)₆(OH)₂, tricalcium phosphate Ca₃(PO₄)₂, calcium monohydrogen phosphate CaHPO₄.(0-2)H₂O, calcium diphosphate Ca₂P₂O₇, octacalcium phosphate Ca₈H₂(PO₄)₆.5H₂O, tetracalcium phosphate Ca₄(PO₄)₂O, or amorphous calcium phosphate Ca₃(PO₄)₂.nH₂O, to convert ethanol to higher molecular weight alcohols are disclosed in WO2006059729.

Hydrotalcites have also been studied as catalysts for making butanol from ethanol. For example, J. I. DiCosimo, et al. discloses the use of Mg_{y}AlOₓ catalysts for alcohol reactions, including ethanol. Journal of Catalysis (1998), 178, 499-510; Journal of Catalysis (2000), 190, 261-275; and Journal of Catalysis (2003) 215, 220-233. U.S. Pat. Nos. 7,705,192 and 7,700,810 disclose the use of partially or fully thermally decomposed hydrotalcites for the conversion of ethanol to butanol. U.S. Pat. No. 7,700,812 discloses the incorporation of the anion of ethylenediaminetetraacetic acid with hydrotalcites for the conversion of ethanol to isobutanol and butanol, respectively. U.S. Pat. No. 7,700,811 discloses hydrotalcite/metal carbonate combinations for the conversion of ethanol to butanol.

Carlini et al., Journal of Molecular Catalysis A: hemical (2005), 232, 13-20, discloses bifunctional heterogeneous hydrotalcites for converting methanol and n-propanol to isobutyl alcohol.

Others catalyst systems for making higher molecular weight alcohols from methanol or ethanol have also been studied. For example, U.S. Pat. No. 4,551,444 discusses the use of multi-component catalyst system using various metals; U.S. Pat. Nos. 5,095,156 and 5,159,125 discuss the impact of magnesium oxide; U.S. Pat. No. 4,011,273 discusses the use of insoluble lead catalysts; U.S. Pat. No. 7,807,857 focuses on Group II metal salts; and U.S. Pat. No. 4,533,775 discusses a catalyst system comprising a metal acetylide, a hydride, an alkoxide and promoter.

The references mentioned above are hereby incorporated by reference.

As such, the need remains for improved catalysts for making butanol from ethanol, especially those having improved activity and selectivity to butanol.

### SUMMARY OF THE INVENTION

In a first embodiment, the present invention is directed to a process for producing a catalyst composition for converting ethanol to butanol. The process comprises the steps of contacting magnesium carbonate with one or more metal precursors. Preferably, the one or more metal precursors comprise one or more metals selected from the group consisting of nickel, palladium, platinum, germanium, copper, ruthenium, gallium, tin, iridium, and mixtures thereof to form a catalyst intermediate. The process further comprises the step of calcining the catalyst intermediate to form the catalyst composition that comprises the metal and magnesium oxide. Preferably, each of the one or more metal precursors is coated on a surface of the magnesium carbonate. Preferably, the contacting step comprises impregnating the magnesium carbonate with the one or more metal precursors. In one embodiment, the one or more metal precursors are selected from the group consisting of metal citrate, metal carboxylate, metal acetate, metal oxalate, and metal acetylacetonate.

In a second embodiment, the invention is directed to a catalyst composition for converting ethanol to butanol. The catalyst comprises from 70 wt. % to 99.9 wt. % magnesium oxide and from 0.1 wt. % to 30 wt. % metal. In one embodiment, the catalyst comprises the formula M-MgO, wherein M is one or more metals selected from the group consisting of nickel, palladium, platinum, germanium, copper, and ruthenium. Preferably, M is selected from the group consisting of palladium, palladium/germanium, palladium/germanium/nickel, palladium/germanium/copper, and palladium/germanium/ruthenium. In one embodiment, the catalyst composition has a surface area from 1 to 800 m²/g. In one embodiment, the catalyst composition has an average pore volume from 0.05 to 3 cm³/g and an average pore diameter from 1 to 10 nm, as determined by BET analysis.

In a third embodiment, the invention is directed to a process for producing butanol. The process comprises the steps of feeding a gaseous stream comprising ethanol over a catalyst composition in a reactor to form butanol, wherein the catalyst composition comprises a magnesium oxide with one or more metals. Preferably, the one or more metals are selected from the group consisting of nickel, palladium, platinum, germanium, copper, and ruthenium. Preferably, the catalyst is formed during calcinations of a metal coated magnesium carbonate and has a high surface and mesoporosity.

In a fourth embodiment, the present invention is to a composition for converting alcohols to higher alcohols, the catalyst is of the formula: Mₐ-M′_{b}-M″_{c}-MgO, wherein M is palladium; M′ is germanium; and M″ is nickel, platinum, ruthenium, and copper; and a is 0.001 to 1, b is 0 to 0.5, and c is 0 to 0.5.

### DETAILED DESCRIPTION OF THE INVENTION

### Introduction

The present invention generally relates to a process for synthesizing a linear multi-carbon alcohol from an alcohol having two or fewer carbons that is useful as a chemical industry raw material and fuel composition or a mixture thereof.

Production of multi-carbon alcohols, like butanol, using most conventional processes has been limited by economic and environmental constraints. One of the best known processes is the Guerbet reaction. Specifically, ethanol may be used as the starting material to product butanol. However, intermediates of the reaction can form competing by-products, such as diethyl ether, ethylene, 1-hexanol, 2-ethylbutanol, and/or 2-ethylhexanol. These intermediates may lead to impurities in the butanol product. For example, diethyl ether and ethylene may be formed due to the dehydration of ethanol in the presence of an acidic catalyst. 1-hexanol may also be formed via the addition of aldehyde to butyraldehyde, a crotonaldehyde intermediate. Butyraldehyde may also react with other intermediates to form 2-ethylbutanol and 2-ethylhexanol. A crude mixture of the multi-carbon alcohol and impurities may increase the purification needed to recover butanol.

The Guerbet reaction converts two moles of ethanol to one mole of butanol through multiple intermediates. The reaction comprising first oxidizing ethanol to form an aldehyde, condensing the aldehydes to 3-hydroxy-butyraldehyde, dehydrating the 3-hydroxy-butyraldehyde to crotonaldehyde, and reducing the crotonaldehyde to butanol.

It has now been discovered that certain catalysts effectively oxidizes ethanol to form an intermediate aldehyde, which forms crotonaldehyde, and reduces crotonaldehyde to butanol. Preferably, the catalysts of the present invention serve as a base to oxidize ethanol and to promote aldol condensation, and also as a hydrogenating site for crotonaldehyde to form butanol. Surprisingly and unexpectedly, the inventors found that a catalyst system of one or more metals coated magnesium oxide made using magnesium carbonate beneficially results in the improvement of ethanol conversion, butanol selectivity and C₄₊ alcohol selectivity. Preferably, the one or more metals are selected from the group consisting of nickel, palladium, platinum, germanium, copper, ruthenium, gallium, tin, iridium, and mixtures thereof. The one or more metals are coated on magnesium carbonate to form a catalyst intermediate and the catalyst intermediate is calcined to form the metal coated magnesium oxide. Surprisingly and unexpectedly, the inventors discovered that coating magnesium carbonate with one or more metals to form the metal coated magnesium oxide produced a catalyst having a greater surface area and pore size than simply coating magnesium oxide with the one or more metals.

### Catalyst Composition

In one embodiment, the present invention is to a catalyst composition for converting alcohols, such as ethanol, to higher alcohols, such as butanol. The catalyst composition comprises magnesium oxide and one or metal precursors. The one or more metal precursors comprise metals selected from the group consisting of nickel, palladium, platinum, germanium, copper, ruthenium, gallium, tin, iridium and mixtures thereof. Preferably, the one or more metals are selected from the group consisting of nickel, palladium, platinum, germanium, copper, and ruthenium. Various combinations of metals may be used to convert ethanol to butanol. For example, metal combinations may include palladium/germanium, nickel/palladium/germanium, copper/palladium/germanium, platinum/palladium/germanium, and ruthenium/palladium/germanium.

In one embodiment, the catalyst comprises a metal-containing magnesium oxide. For example, in one embodiment, the catalyst comprises from 70 wt. % to 99.9 wt. % magnesium oxide and from 0.1 wt. % to 30 wt. % metal, e.g., comprises from 75 wt. % to 99 wt. % magnesium oxide and 1 wt. % to 20 wt. % metal, or comprises from 80 wt. % to 98 wt. % magnesium oxide and 2 wt. % to 10 wt. % metal.

In one embodiment, the magnesium carbonate is contacted with one or more metals (M) selected from the group consisting of consisting of nickel, palladium, platinum, germanium, copper, ruthenium, gallium, tin, iridium and mixtures thereof. In one embodiment, the one or more metals are contacted with magnesium carbonate to form an intermediate catalyst. The intermediate catalyst is calcined to form the metal-coated magnesium oxide. In one embodiment, the catalysts may be a multi-layer metal-coated magnesium oxide. As such, one or more layers of metals may be contacted with magnesium carbonate and calcined to form the metal-coated magnesium oxide catalyst. In one embodiment, the one or more layers of metals are different metals. In one embodiment, the different layers of metals are intimately contacted with magnesium carbonate. In one embodiment, different metals may be mixed together prior to contacting magnesium carbonate.

“Coat,” “coated,” or “coating” as used in the present application generally refers to one or more metals distributed on the surface of magnesium carbonate during the catalyst process and those metals remain distributed on the surface when calcined to magnesium oxide.

The catalyst may be represented by the formula: M-MgO. In one embodiment, the catalyst may comprise magnesium oxide and metals selected from the group consisting of nickel, palladium, platinum, germanium, copper, ruthenium, gallium, tin, iridium and mixtures thereof. In another embodiment, the catalyst may comprise palladium and magnesium oxide and the M-MgO composition comprises from 0.01 wt % to 10 wt. % palladium, e.g., from 0.05 wt. % to 8 wt. %, or from 0.1 wt. % to 5 wt. %. In yet another embodiment, the catalyst may comprise germanium and magnesium oxide and the M-MgO composition comprises from 0.1 wt% to 20 wt. % germanium, e.g., from 0.5 wt. % to 18 wt. %, or from 1 wt. % to 15 wt. %. In yet another embodiment, the catalyst may comprise nickel and magnesium oxide and the M-MgO composition comprises from 0.01 wt. % to 20 wt. % nickel, e.g., from 0.1 wt. % to 18 wt. %, or from 0.5 wt. % to 15 wt. %. In yet another embodiment, the catalyst may comprise ruthenium and magnesium oxide and the M-MgO composition comprises from 0.01 wt% to 10 wt. % ruthenium, e.g., from 0.05 wt. % to 8 wt. %, or from 0.1 wt. % to 7 wt. %. In yet another embodiment, the catalyst may comprise copper and magnesium oxide and the M-MgO composition comprises from 0.01 wt% to 20 wt. % copper, e.g., from 0.1 wt. % to 18 wt. %, or from 0.5 wt. % to 15 wt. %.

In another embodiment, the catalyst comprises magnesium oxide with two metals (M and M′) selected from the group consisting of nickel, palladium, platinum, germanium, copper, ruthenium, gallium, tin, iridium and mixtures thereof. The two metals coated magnesium oxide catalyst may be represented by the formula: M-M′-MgO. In one embodiment, the catalyst may comprise palladium, germanium and magnesium oxide and the M-M′-MgO composition comprises from 0.01 wt. % to 10 wt. % palladium, e.g., from 0.05 wt. % to 8 wt. %, or from 0.1 wt. % to 5 wt. %; from 0.1 wt. % to 20 wt. % germanium, e.g., from 0.5 wt. % to 18 wt. %, or from 1 wt. % to 15 wt. %; and from 70 wt. % to 99.89 wt. % magnesium oxide, e.g., from 74 wt. % to 99.45 wt. %, or from 80 wt. % to 98.9 wt. %.

In yet another embodiment, the catalyst may comprise magnesium oxide with three metals selected from the group consisting of nickel, palladium, platinum, germanium, copper, ruthenium, gallium, tin, iridium and mixtures thereof. The two metals coated magnesium oxide catalyst may be represented by the formula: M-M′-M″-MgO. The magnesium oxide with three metals catalyst may be represented by the formula: M-M′-M″-MgO.

In one embodiment, the catalyst may comprise nickel, palladium, germanium and magnesium oxide. The M-M′-M″-MgO composition comprises from 0.01 wt. % to 20 wt. % nickel, e.g., from 0.1 wt. % to 18 wt. %, or from 0.5 wt. % to 15 wt. %; from 0.01 wt. % to 10 wt. % palladium, e.g., from 0.05 wt. % to 8 wt. %, or from 0.1 wt. % to 5 wt. %; and from 0.01 wt. % to 20 wt. % germanium, e.g., from 0.5 wt. % to 18 wt. %, or from 1 wt. % to 15 wt. %.

Another exemplary multiple metal catalyst of the present invention comprises copper, palladium, germanium and magnesium oxide. In one embodiment, the M-M′-M″-MgO composition comprises from 0.01 wt. % to 20 wt. % copper, e.g., from 0.1 wt. % to 18 wt. %, or from 0.5 wt. % to 15 wt. %; from 0.01 wt. % to 10 wt. % palladium, e.g., from 0.05 wt. % to 8 wt. %, or from 0.1 wt. % to 5 wt. %; and from 0.1 wt. % to 20 wt. % germanium, e.g., from 0.5 wt. % to 18 wt. %, or from 1 wt. % to 15 wt. %.

Another exemplary multiple metal catalyst of the present invention comprises ruthenium, palladium, germanium and magnesium oxide. In one embodiment, the M-M′-M″-MgO composition comprises from 0.01 wt. % to 10 wt. % ruthenium, e.g., from 0.05 wt. % to 8 wt. %, or from 0.1 wt. % to 7 wt. %; from 0.01 wt. % to 10 wt. % palladium, e.g., from 0.05 wt. % to 8 wt. %, or from 0.1 wt. % to 5 wt. %; and from 0.1 wt. % to 20 wt. % germanium, e.g., from 0.5 wt. % to 18 wt. %, or from 1 wt. % to 15 wt. %.

Another exemplary multiple metal catalyst of the present invention comprises platinum, palladium, germanium and magnesium oxide. In one embodiment, the M-M′-M″-MgO composition comprises from 0.01 wt. % to 10 wt. % platinum, e.g., from 0.05 wt. % to 8 wt. %, or from 0.1 wt. % to 5 wt. %; from 0.01 wt. % to 10 wt. % palladium, e.g., from 0.05 wt. % to 8 wt. %, or from 0.1 wt. % to 5 wt. %; and from 0.1 wt. % to 20 wt. % germanium, e.g., from 0.5 wt. % to 18 wt. %, or from 1 wt. % to 15 wt. %.

In one embodiment, the catalyst corresponds to the formula:

Mₐ-M′_{b}-M″_{c}-MgO

wherein the letters a, b, and c are the relative molar amounts (relative to 1) of magnesium oxide, first metal (M), second metal (M′) and third metal (M″), respectively in the catalyst. Preferably, M is palladium. Preferably, M′ is germanium. In these embodiments, M′ or M″ may not be present in the catalyst. In an embodiment, M″ is nickel, platinum, ruthenium or copper. In one embodiment, a is 0.001 to 0.1, and b, and c are each independently 0 to 0.5.

It has now been found that the catalyst system of one or more metals coated magnesium oxide made using magnesium carbonate beneficially results in the improvement of ethanol conversion, butanol selectivity and C₄₊ alcohol selectivity than simply coating magnesium oxide with one or more metals. For example, depending on the temperature and pressure at which the Guerbet reaction is conducted, ethanol conversion of at least 20%, e.g., at least 30%, or at least 40%, may be achieved with the catalyst compositions. Surprisingly and unexpectedly, this increase of ethanol conversion is achieved with improved selectivity to butanol. For example, selectivity to butanol of at least 30%, e.g., at least 40%, or at least 50%. Without being bound by theory, it is postulated that the catalysts of the present invention serve as a base to oxidize ethanol and promote aldol condensation and also as a hydrogenating site for crotonaldehyde to form butanol.

As stated above the metal-coated magnesium oxide is made by contacting one or more metals with magnesium carbonate to form an intermediate catalyst. The intermediate catalyst is calcined to form the metal-coated magnesium oxide that has a larger surface area, average pore volume and average pore diameter than metal-coated directly onto magnesium oxide. In one embodiment, surprisingly and unexpectedly, it has now been found that the catalyst beneficially enhance ethanol conversion, butanol selectivity, and C₄₊ alcohols selectivity. It is believed that by contacting one or more metals with magnesium carbonate and calcining such surprisingly and unexpectedly results in a final catalyst that has larger surface area, average pore volume and average pore diameter. It is also postulated that the metals are more intimately contacted with magnesium carbonate and the interaction between the metal and the support is stronger than simply coating magnesium oxide with metals.

Water is a byproduct when converting ethanol to butanol. Since water is more polar than ethanol, it is believed that water might compete with ethanol on the polar surface of the catalyst. The inventors have found that the surface polarity of the catalysts may be modified by introducing an organic metal precursor to the surface of the support to minimize the water/ethanol competition. The organic metal precursor may include pyridine, ammonium hydroxide tetramethylammonium hydroxide, tetrabutylammonium hydroxide, methyl amine, imidazole, and other suitable support modifiers. The organic metal precursors may be support modifiers that may adjust the chemical or physical properties of the support material such as the acidity or basicity of the support material. For purposes of the present invention, the support material is magnesium oxide. As such, the amount and residence time of ethanol on the surface of the catalyst maybe increased and thereby promoting the carbon-carbon capillary condensation.

In other embodiments, in addition to a support, the catalyst may further comprise a support modifier. A modified support, in one embodiment, relates to a support that includes a support material and a support modifier, which, for example, may adjust the chemical or physical properties of the support material such as the acidity or basicity of the support material.

In some embodiments, the catalyst may comprise other metal(s) and/or metal precursors. For example, lithium, sodium, potassium, rubidium, cesium, francium, unununium, calcium, strontium, barium, lanthanum, cerium, and other suitable metals and/or metal precursors. In some embodiments, the catalyst optionally may comprise additional metals and/or metal precursors in an amount from 0.001 wt. % to 30 wt. %, e.g., from 0.01 wt. % to 5 wt. % or from 0.1 wt. % to 5 wt. %.

In some embodiments, the catalyst may have high surface area, e.g., the catalyst may have a surface area of at least 1 m²/g, e.g., at least 20 m²/g or at least 50 m²/g, as determined by BET measurements. The catalyst may include pores, optionally having an average pore diameter ranging from 2 nm to 200 nm, e.g., from 3 nm to 50 nm or from 5 nm to 30 nm. The catalyst has an average pore volume of from 0.05 cm³/g to 3 cm³/g, e.g., from 0.08 cm³/g to 2 cm³/g or from 0.08 cm³/g to 1 cm³/g, as determined by BET measurements. Preferably, at least 50% of the pore volume or surface area, e.g., at least 70% or at least 80%, is provided by pores having the diameters discussed above. Pores may be formed and/or modified by pore modification agents, which are discussed below. Mesoporosity refers to pores greater than 2 nm and less than 50 nm is diameter. Flow through mesopores may be described by Knudson diffusion. Macroporosity refers to pores greater than 50 nm in diameter and flow though macropores may be described by bulk diffusion. Thus, in some embodiments, it is desirable to balance the surface area, pore size distribution, catalyst or support particle size and shape, and rates of reaction with the rate of diffusion of the reactant and products in and out of the pores to optimize catalytic performance.

The catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In some embodiments, the catalyst composition comprises a pore modification agent, such as oxalic acid. A preferred type of pore modification agent is thermally stable and has a substantial vapor pressure at a temperature below 300° C., e.g., below 250° C. In one embodiment, the pore modification agent has a vapor pressure of at least 0.1 kPa, e.g., at least 0.5 kPa, at a temperature between 150° C. and 250° C., e.g., between 150° C. and 200° C.

The pore modification agent has a relatively high melting point, e.g., greater than 60° C., e.g., greater than 75° C., to prevent melting during the compression of the catalyst into a slug, tablet, or pellet. Preferably, the pore modification agent comprises a relatively pure material rather than a mixture. As such, lower melting components will not liquefy under compression during formation of slugs or tablets. For example, where the pore modification agent is a fatty acid, lower melting components of the fatty acid mixtures may be removed as liquids by pressing. If this phenomenon occurs during slug or tablet compression, the flow of liquid may disturb the pore structure and produce an undesirable distribution of pore volume as a function of pore diameter on the catalyst composition. In other embodiments, the pore modification agents have a significant vapor pressure at temperatures below their melting points, so that they can be removed by sublimination into a carrier gas.

### Catalyst Preparation

Unlike previously disclosed metal-coated magnesium oxide where the metals are coated on the magnesium oxide, the process for preparing the catalyst of the present invention involves contacting one or more metals with magnesium carbonate to form an intermediate catalyst and calcining it to form the metal-coated magnesium oxide. The catalysts of the present invention may be synthesized by the following methods.

Magnesium carbonate is measured and pressed, under force, for a predetermined time to form pellets. The pellets are lightly crushed to a desired particle size. An amount of the magnesium carbonate pellets is measured and placed in a round bottom reactor. A metal precursor, such as a metal acetate or a metal nitrate, may be dissolved in an amount of solvent, such as water, ethanol, or acetone to form a colloidal dispersion of the metal precursor. The metal precursor solution is impregnated onto the magnesium carbonate pellets by stepwise incipient wetness using a rotating dryer. The metal coated magnesium carbonate is dried overnight at a desired temperature for a period of time and followed by calcinations to form the catalyst with one metal coating.

In one embodiment, a multi-layered metal-coated magnesium oxide may be prepared by adding a second or a third metal layer to the metal-coated magnesium oxide. For example, the metal-coated magnesium oxide may be cooled after calcinations and a second metal nitrate or metal oxide may be coated thereon. The twice-coated magnesium oxide may be dried and calcined before a third metal layer is added.

In one embodiment, any suitable metal precursors may be used to make the catalyst composition. Non-limiting examples of suitable metal precursors include metal salts of inorganic and organic acids such as, e.g., citrates, carboxylates, acetate, oxalates, and acetylacetonates and salts and acids wherein the metal is part of an anion. For purposes of the present invention at least one of the metal precursors include the citrates, carboxylates, acetate, oxalates, and acetylacetonates. In other embodiments, the second or third metal precursors may be selected from the group consisting of nitrites, nitrates, oxides, sulfates, halides (e.g., fluorides, chlorides, bromides and iodides), carbonates, phosphates, azides, borates (including fluoroborates, pyrazolylborates, etc.), sulfonates, substituted carboxylates (including halogenocarboxylates such as, e.g., trifluoroacetates, hydroxycarboxylates, aminocarboxylates, etc.) and salts and acids wherein the metal is part of an anion (such as, e.g., hexachloroplatinates, tetrachloroaurate, tungstates and the corresponding acids).

Further non-limiting examples of suitable metal precursors for the processes of the present invention include alkoxides, complex compounds (e.g., complex salts) of metals such as, e.g., beta-diketonates (e.g., acetylacetonates), complexes with amines, N-heterocyclic compounds (e.g., pyrrole, aziridine, indole, piperidine, morpholine, pyridine, imidazole, piperazine, triazoles, and substituted derivatives thereof), aminoalcohols (e.g., ethanolamine, etc.), amino acids (e.g., glycine, etc.), amides (e.g., formamides, acetamides, etc.), and nitriles (e.g., acetonitrile, etc.). Non-limiting examples of preferred metal precursors include nitrates and oxides.

Non-limiting examples of specific metal precursors for use in the processes of the present invention include germanium oxide, germanium butoxide, germanium glycolate, germanium chloride, germanium acetate, germanium hydroxide, germanium methoxide, geimanium nitride, and bis(2-carboxyethyl germanium sesquioxide); palladium acetate, palladium bromide, palladium chloride, palladium iodide, palladium nitrate, palladium nitrate hydrate, tetraamine palladium nitrate, palladium oxide, palladium oxide hydrate, and palladium sulfate; copper oxide, copper hydroxide, copper nitrate, copper sulfate, copper chloride, copper formate, copper acetate, copper neodecanoate, copper ethylhexanoate, copper methacrylate, copper trifluoroacetate, copper acetoacetate and copper hexafluoroacetylacetonate; platinum formate, platinum acetate, platinum propionate, platinum benzoate, platinum stearate, platinum neodecanoate, and platinum(diaminopropane)(ethylhexanoate). The above compounds may be employed as such or optionally in the form of solvates and the like such as, e.g., as hydrates. Examples of specific metal precursors that may be used in the present invention include palladium acetate, germanium oxide, bis(2-carboxyethyl germanium sesquioxide), palladium nitrate hydrate, copper nitrate hydrate, nickel nitrate hydrate, and platinum acetate.

The use of mixtures of different compounds, e.g., different salts, of the same metal and/or the use of mixtures of compounds of different metals and/or of mixed metal precursors (e.g., mixed salts and/or mixed oxides) is also contemplated by the present invention. Accordingly, the term “metal precursor” as used herein includes both a single metal precursor and any mixture of two or more metal precursors. In a preferred embodiment, the catalyst composition is made using magnesium oxide and a first metal precursor, and optionally a second metal precursor and a third metal precursor. The first metal precursor is a precursor to a first metal, the second metal precursor is a precursor to a second metal, and the third metal precursor is a precursor to a third metal.

### Production of Butanol

Suitable reactions and/or separation scheme may be employed to form a crude product stream comprising butanol using the catalysts. For example, in some embodiments, the crude product stream is formed by contacting a low molecular weight alcohol, e.g., ethanol, with the catalysts to form the crude higher alcohol product stream, i.e., a stream with butanol. Preferably, the catalyst is a metal-coated magnesium oxide. In a preferred embodiment, the crude product stream is the reaction product of the condensation reaction of ethanol, which is conducted over a metal-coated magnesium oxide. In one embodiment, the crude product stream is the product of a vapor phase reaction.

In some embodiments, the condensation reaction may achieve favorable conversion of ethanol and favorable selectivity and productivity to butanol. For purposes of the present invention, the term “conversion” refers to the amount of ethanol in the feed that is converted to a compound other than ethanol. Conversion is expressed as a percentage based on ethanol in the feed. The conversion of ethanol may be at least 20%, e.g., at least 30%, at least 40%, or at least 60%.

The feedstream may be a gaseous stream comprising ethanol. Preferably, the gaseous stream comprise more than 5 vol.% ethanol, e.g., more than 10 vol.% or more than 20 vol.%. The feed stream may also comprise other molecules such as pyridine, NH₃, and alkyl amine. Inert gases may be in the gaseous stream and thus may include nitrogen, helium, argon, and methane. Preferably, no hydrogen is introduced with the gaseous stream, and thus the gaseous stream is substantially free of hydrogen. Without being bound by theory the hydrogen needed for the intermediate reactions may be produced in situ.

Selectivity, as it refers to the formation of butanol, is expressed as the ratio of the amount of carbon in the desired product(s) and the amount of carbon in the total products. This ratio may be multiplied by 100 to arrive at the selectivity. Preferably, the selectivity to butanol is at least 30%, e.g., at least 40%, or at least 60%. In some embodiments, the catalyst selectivity to C₄₊ alcohols, e.g., n-butanol, isobutanol, 2-butanol, or tert-butanol, is at least 30%, e.g., at least 50%, at least 60%, or at least 80%.

The ethanol may be fed to the reactor as a liquid stream or a vapor stream. Preferably, the ethanol is fed as a vapor stream. The reactor may be any suitable reactor or combination of reactors. Preferably, the reactor comprises a fixed bed reactor or a series of fixed bed reactors. In one embodiment, the reactor is a gas flow catalytic reactor or a series of gas flow catalytic reactors. Of course, other reactors such as a continuous stirred tank reactor or a fluidized bed reactor, may be employed.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 0.01 to 100 hours, e.g., from 1 to 80 hours, or from 5 to 80 hours. Reaction pressure is not particularly limited, and the reaction is typically performed near atmospheric pressure. In one embodiment, the reaction may be conducted at a pressure ranging from 0.1 kPa to 9,000 kPa, e.g., from 20 kPa to 5,000 kPa, or from 90 to 3,500 kPa. The ethanol conversion, in some embodiments, may vary depending upon the reaction temperature and/or pressure.

In one embodiment, the reaction is conducted at a gas hourly space velocity (“GHSV”) greater than 600 hr⁻¹, e.g., greater than 1,000 hr⁻¹ or greater than 2000 hr⁻¹. The GHSV may range from 600 hr⁻¹ to 10,000 hr⁻¹, e.g., from 1,000 hr⁻¹ to 8,000 hr^{-‘}or from 1,500 hr⁻¹ to 7,500 hr⁻¹.

In one embodiment, an inert or reactive gas is supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors. The addition of these additional components may improve reaction efficiencies.

In one embodiment, the unreacted components such as the ethanol as well as the inert or reactive gases that remain are recycled to the reactor after sufficient separation from the desired product.

### EXAMPLES

### Example 1

### Palladium Magnesium Oxide (Pd—MgO) Using Magnesium Carbonate

10 g of MgCO₃ was measured and pressed at 180,000 N for 1 hour to form pellets. The pellets were then lightly crushed to a particle size of 0.85 mm and 1.18 mm. 0.127 g of palladium acetate was dissolved in 10 g of acetone and a few drops of ethanol (˜0.5 ml) were added to allow uniform colloidal dispersion. The clear colloid was impregnated to the above pre-shaped MgCO₃ by stepwise incipient wetness using the rotating dryer. The catalyst was dried slowly overnight in air (50° C./ramp 0.5° C./min). The catalyst was then dried for 8 hours at 120° C. (ramp 2° C./min). The catalyst was calcined at 425° C. for 4 hours (ramp 0.5 ° C./min), followed by cooling to room temperature.

### COMPARATIVE EXAMPLE

### Palladium Magnesium Oxide (Pd—MgO) Using Magnesium Oxide

MgO (extrudate) was crushed to a particle size of 0.85 mm and 1.18 mm. 0.155 g of palladium nitrate was dissolved in 5 g of H₂O, followed by impregnating to the above pre-shaped MgO by stepwise incipient wetness using the rotating dryer. The obtained sample was dried in the oven at 120° C. for 5 hours, followed by calcination using the following temperature program: start at 60° C., ramp to 500° C. at 2° C./min, hold at 500° C. for 5 hours, followed by cooling to room temperature.

### Example 2

The above-prepared catalysts were evaluated. A fixed bed gas flow catalytic reactor was used as a reactor. 3 ml of above prepared catalysts was filled in a stainless steel tube reactor with a diameter of 0.95 cm. As a pretreatment, hydrogen reduction was conducted for 1 hour under a carrier gas atmosphere (10% H₂/N₂ base; flow rate 125 ml/min) at 400° C. After the pretreatment, the testing was conducted at a temperature between 250° C. and 325° C. and pressure between 1 kPa and 5,100 kPa, nitrogen flow rate was at 125 sccm and ethanol flow rate was at 0.2 ml/min. The reaction duration ranges from 5 hours to 80 hours.

Catalytic performance of Pd—MgO using above two methods were compared. The ethanol conversion, product selectivity, yield, and C₄₊ alcohol selectivity for the catalysts are shown below in Table 1.

TABLE 1
Pd—MgO using MgCO₃ vs. Pd—MgO using MgO
Testing condition: 250° C. and 3,400 kPa

| | Ethanol | Butanol | | C₄₊ Alcohols |
|---|---|---|---|---|
| | Conversion | Selectivity | Yield | Selectivity |
| Catalysts | (%) | (%) | (%) | (%) |
| Example 1 | 41 | 77 | 19 | 93 |
| Comparative | 3.9 | 37 | 1.4 | 68 |
| Example | | | | |

As shown in Table 1, Example 1 (palladium magnesium oxide made using magnesium carbonate) has a significant better ethanol conversion, butanol selectivity, yield and C₄₊ alcohols selectivity than the comparative example (palladium magnesium oxide made using magnesium oxide). For example, Example 1 has an ethanol conversion of 41%, a butanol selectivity of 77%, a yield of 19%, and a C₄₊ alcohol selectivity of 93%. In comparison, Comparative Example has an ethanol conversion of 3.9%, a butanol selectivity of 37%, a yield of 1.4%, and a C₄₊ alcohol selectivity of 68%.

### Example 3

### Palladium (0.6 wt. %)-Germanium (2.1 wt. %) Magnesium Oxide (Pd—Ge—MgO)

The catalyst was synthesized using sequential impregnation method with Ge on the inner layer and Pd on the outer layer. 10 g of the crushed compressed MgCO₃, prepared as in Example 1, was measured and placed in a round bottom reactor. 0.55 g of Bis(2-carboxyethylGermanium (IV) sesquioxide) was dispersed in 6 g of distilled H₂O. 1.1 g of oxalic acid hydrate was dissolved in 2 g of distilled water, and then slowly added to the above Ge suspension. The resulted mixture was then heated at 50° C. until the solution turns clear; followed by impregnating to the MgCO₃ by stepwise incipient wetness using the rotating dryer. The obtained sample was dried in the oven at 120° C. for 8 hours, followed by calcination using the following temperature program: start at 60° C., ramp to 425° C. at 2° C./min, hold at 425° C. for 4 hours, followed by cooling to room temperature. 0.127 g of palladium acetate was dissolved in 10 g of acetone and a few drops of ethanol (˜0.5 ml) were added to allow uniform colloidal dispersion. The clear colloid was impregnated to the above Ge treated MgCO₃ by stepwise incipient wetness using the rotating dryer. The catalyst was slow dried overnight in air (50° C./ramp 0.5° C./min). The catalyst was then dried for 8 hours at 120° C. (ramp 2° C./min). The catalyst was calcined at 425° C. for 4 hours (ramp 0.5° C./min), followed by cooling to room temperature.

### Example 4

### Nickel (1 wt. %)-Palladium (0.6 wt. %)-Germanium (2.1 wt. %) Magnesium Oxide (Ni—Pd—Ge—MgO)

The catalyst was synthesized using sequential impregnation method with Ge on the inner layer, Pd on the medium layer and Ni on the outer layer. The pretreated support of Example 3 was impregnated with 0.377 g of Ni(NO₃)₂.2H₂O was dissolved in 5 g of H₂O by stepwise incipient wetness using the rotating dryer. The obtained sample was dried in the oven at 120° C. for 8 hours, followed by calcination using the following temperature program: start at 60° C., ramp to 500° C. at 2° C./min, hold at 500° C. for 5 hours, followed by cooling to room temperature.

### Example 5

### Nickel (0.05 wt. %)-Palladium (0.6 wt. %)-Germanium (2.1 wt. %) Magnesium Oxide (Ni—Pd—Ge—MgO)

The catalyst was synthesized using sequential impregnation method with Ge on the inner layer, Pd on the medium layer and Ni on the outer layer. The pretreated support of Example 3 was impregnated with 0.0187 g of Ni(NO₃)₂.2H₂O was dissolved in 5 g of H₂O, by stepwise incipient wetness using the rotating dryer. The obtained sample was dried in the oven at 120° C. for 8 hours, followed by calcination using the following temperature program: start at 60° C., ramp to 500° C. at 2° C./min, hold at 500° C. for 5 hours, followed by cooling to room temperature.

### Example 6

### Copper (1 wt. %)-Palladium (0.6 wt. %)-Germanium (2.1 wt. %) Magnesium Oxide (Cu—Pd—Ge—MgO)

The catalyst was synthesized using sequential impregnation method with Ge on the inner layer, Pd on the medium layer and a second Pd on the outer layer. The pretreated support of Example 3 was impregnated with 0.356 g of Cu(NO₃)₂.2H₂O was dissolved in 5 g of H₂O, by stepwise incipient wetness using the rotating dryer. The obtained sample was dried in the oven at 120° C. for 8 hours, followed by calcination using the following temperature program: start at 60° C., ramp to 500° C. at 2° C./min, hold at 500° C. for 5 hours, followed by cooling to room temperature.

### Example 7

### Platinum (0.05 wt. %)-Palladium (0.6 wt. %)-Germanium (2.1 wt. %) Magnesium Oxide (Pt—Pd—Ge—MgO)

The catalyst was synthesized using sequential impregnation method with Ge on the inner layer, Pd on the medium layer and Pt on the outer layer. The pretreated support of Example 3 was impregnated with 0.0005 g of Pt(OAc)₂.2H₂O was dissolved in 5 g of H₂O, by stepwise incipient wetness using the rotating dryer. The obtained sample was dried in the oven at 120° C. for 8 hours, followed by calcination using the following temperature program: start at 60° C., ramp to 350° C. at 2° C./min, hold at 350° C. for 5 hours, followed by cooling to room temperature.

### Example 8

### Ruthenium (0.5 wt. %)-Palladium (0.6 wt. %)-Germanium (2.1 wt. %) Magnesium Oxide (Ru—Pd—Ge—MgO)

The catalyst was synthesized using sequential impregnation method with Ge on the inner layer, Pd on the medium layer and Ru on the outer layer. The pretreated support of Example 3 was impregnated with 0.158 g of ruthenium(III) nitrosyl nitrate was dissolved in 5 g of H₂O, by stepwise incipient wetness using the rotating dryer. The obtained sample was dried in the oven at 120° C. for 8 hours, followed by calcination using the following temperature program: start at 60° C., ramp to 500° C. at 2° C./min, hold at 500° C. for 5 hours, followed by cooling to room temperature.

### Example 9

The above-prepared catalysts were evaluated using the procedure in Example 2. The ethanol conversion, product selectivity, yield, and C₄₊ alcohol selectivity for the above catalysts are shown below in Table 2.

TABLE 2
Comparisons of Metal Coated MgO
Testing condition: 250° C. and 3,400 kPa

| | Ethanol | Butanol | Yield | C₄₊ Alcohols |
|---|---|---|---|---|
| Catalysts | Conversion (%) | Selectivity (%) | (%) | Selectivity (%) |
| Comparative | 3.9 | 37 | 1.4 | 68 |
| Example | | | | |
| Example 1 | 41 | 77 | 32 | 93 |
| Example 3 | 32 | 69 | 24 | 83 |
| Example 4 | 40 | 83 | 33 | 92 |
| Example 5 | 39 | 80 | 31 | 90 |
| Example 6 | 52 | 67 | 35 | 81 |
| Example 7 | 45 | 80 | 36 | 92 |
| Example 8 | 21 | 67 | 14 | 82 |

Examples 1 and 3-8 were made using magnesium carbonate. As shown in Table 2, Examples 1 and 3-8 show greater ethanol conversion, butanaol selectivity, yield, and C₄₊ alcohols selectivity than Comparative Example, which is made with magnesium oxide. For example, the one metal coated MgO catalyst (Example 1: Pd—MgO) shows greatly improved ethanol conversion, butanol selectivity, yield and C₄₊ alcohols selectivity. The two metals coated MgO catalyst (Example 3: Pd—Ge—MgO) also shows improved ethanol conversion, butanol selectivity, yield and C₄₊ selectivity. Surprisingly and unexpectedly, when a third metal is added to Pd—Ge—MgO in Examples 4-8, ethanol conversion, butanol, and yield are also greater than the Comparative Example. In each case, the three metals coated MgO catalysts show better performance than the Comparative Example.

Examples 4 and 5 show activity of the catalysts with nickel loading of 1 wt. % and 0.05 wt. %, respectively. It does not appear that the difference in nickel loading has an effect on the activity of the catalyst.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention.

We claim:

1. A process for producing a catalyst composition for converting ethanol to butanol, the process comprising:
contacting magnesium carbonate with one or more metal precursors, wherein the one or more metal precursors comprise one or more metals selected from the group consisting of nickel, palladium, platinum, germanium, copper, ruthenium, gallium, tin, iridium and mixtures thereof to form a catalyst intermediate; and
calcining the catalyst intermediate to faun the catalyst composition comprises the one or more metals and magnesium oxide.

2. The process of claim 1, wherein at least one of the one or more metal precursors is coated on a surface of the magnesium carbonate.

3. The process of claim 1, wherein at least one of the one or more metal precursors is intimately contacted with magnesium carbonate.

4. The process of claim 1, further comprising mixing the one or more metal precursors prior to the contacting step.

5. The process of claim 1, wherein the one or more metal precursors are selected from the group consisting of metal citrate, metal carboxylate, metal acetate, metal oxalate, and metal acetylacetonate.

6. The process of claim 1, wherein the contacting step comprises impregnating the magnesium carbonate with at least one of the one or more metal precursors.

7. The process of claim 1, wherein the calcining step is a temperature from 300° C. to 750° C.

8. A catalyst composition for converting ethanol to butanol produced according to the process of claim 1.

9. The catalyst composition of claim 8, wherein the catalyst comprises from 70 wt. % to 99.9 wt. % magnesium oxide and from 0.1 wt. % to 30 wt. % metal.

10. The catalyst composition of claim 8, wherein the catalyst is of the formula:
M-MgO,
wherein M is one or more metals selected from the group consisting of nickel, palladium, platinum, germanium, copper, and ruthenium.

11. The catalyst composition of claim 10, wherein M is selected from the group consisting of palladium, palladium/germanium, palladium/germanium/nickel, palladium/germanium/copper, and palladium/germanium/ruthenium.

12. The catalyst composition of claim 10, wherein the catalyst comprises from 0.1 wt. % to 10 wt. % palladium.

13. The catalyst composition of claim 10, wherein the catalyst comprises from 0.1 wt. % to 20 wt. % germanium.

14. The catalyst composition of claim 10, wherein the catalyst comprises from 0.1 wt. % to 20 wt. % nickel.

15. The catalyst composition of claim 10, wherein the catalyst comprises from 0.1 wt. % to 10 wt. % ruthenium.

16. The catalyst composition of claim 10, wherein the catalyst comprises from 0.1 wt. % to 20 wt. % copper.

17. The catalyst composition of claim 8, wherein the catalyst composition has a surface area from 1 to 800 m²/g.

18. The catalyst composition of claim 8, wherein the catalyst composition has an average pore volume from 0.05 to 2 cm³/g, as determined by BET analysis.

19. The catalyst composition of claim 8, wherein the catalyst composition has an average pore diameter from 1 to 10 nm, as determined by BET analysis.

20. A process for producing higher alcohols, the process comprising the steps of:
feeding a gaseous stream comprising ethanol over a catalyst composition in a reactor to form butanol, wherein the catalyst composition comprises a magnesium oxide with one or more metals, wherein the one or more metals are selected from the group consisting of nickel, palladium, platinum, gelinanium, copper, and ruthenium, wherein the catalyst is formed during calcination of a metal coated magnesium carbonate and having a high surface area and mesoporosity.

21. The process of claim 20, wherein ethanol conversion is at least 20%.

22. The process of claim 20, wherein butanol selectivity is at least 30%.

23. The process of claim 20, wherein the catalyst comprises from 70 wt. % to 99.9 wt. % magnesium oxide and from 0.1 wt. % to 30 wt. % metal.

24. The process of claim 20, wherein the catalyst is of the formula:
M-MgO, wherein M is selected from the group consisting of nickel, palladium, platinum, germanium, copper, ruthenium, and mixtures thereof.

25. A catalyst composition for converting alcohols to higher alcohols, the catalyst is of the formula:
Mₐ-M′_{b}-M″_{c}-MgO
wherein M is palladium;
M′ is germanium;
M″ is nickel, platinum, ruthenium, or copper; and
a is 0.001 to 0.1,
b is 0 to 0.5, and
c is 0 to 0.5.
